SEQUENCE LISTING (2 sequences)

SEQ ID NO: 1 - RNA, 19 nt, Homo sapiens
  source 1..19: mol_type = other RNA
cggggtcggcggcgacgtg

SEQ ID NO: 2 - DNA, 23 bp, Homo sapiens
  source 1..23: mol_type = genomic DNA
ggcccagcccaaggagaccccac

(19) United States
(12) Patent Application Publication
(10) Pub. No.: US 2024/0167039 A1
(43) Pub. Date: May 23, 2024

(54) METHOD FOR TREATING LUNG INJURY

(71) Applicant: NATIONAL YANG MING CHIAO TUNG UNIVERSITY, Hsinchu (TW)

(72) Inventors: Wei-Ting LIN, Taipei City (TW); Hao-Hsiang WU, Taipei City (TW); Oscar Kuang-Sheng LEE, Taipei City (TW); Jennifer Hui-Chun HO, San Diego, CA (US)

(21) Appl. No.: 18/057,676

(22) Filed: Nov. 21, 2022

(51) Int. Cl. C12N 15/113; A61P 11/00; C12N 5/0775

(52) U.S. Cl. CPC C12N 15/1135; A61P 11/00; C12N 5/0667; C12N 2310/141; C12N 2501/115; C12N 2501/148; C12N 2501/24

(57) ABSTRACT

The present disclosure provides a method for treating and/or ameliorating a lung injury or inflammation in the lung, and/or promoting polarization of macrophages in a subject in need thereof, wherein the method comprises administering microRNA-7704 (miR-7704) or a composition comprising miR-7704 to the subject.

### SEQUENCE LISTING INFORMATION

The instant application contains a Sequence Listing which has been submitted electronically in XML format and is hereby incorporated by reference in its entirety. Said XML copy, created on Nov. 21, 2022, is named “G4590-17000NP_SeqListing_20221121.xml” and is 3 kilobytes in size.

### FIELD OF THE INVENTION

The present disclosure relates to a method for treating a lung disease. Particularly, the present disclosure relates to a method for treating a lung injury.

### BACKGROUND OF THE INVENTION

Lung diseases, including respiratory diseases, are a major cause of mortality and morbidity worldwide. Current treatments are directed to reducing symptoms of lung disease and offer little to no prospect of cure or complete disease reversal. Acute lung injury (ALI), also known as acute respiratory distress syndrome (ARDS), is a life threatening inflammatory disease. ALI is a critical illness leading to permeability pulmonary edema and respiratory failure. The clinical scenarios that place patients at risk for ALI are as diverse as trauma, hemorrhage or sepsis. Despite significant advances in critical care management, mortality from ALI remains high.

However, current therapies for acute lung injury are largely inadequate. Therefore, there remains a need for effective ALI treatment.

### SUMMARY OF THE INVENTION

The present disclosure provides a method for treating and/or ameliorating a lung injury in a subject in need thereof, wherein the method comprises administering an effective amount of isolated microRNA-7704 (miR-7704) or a composition comprising an effective amount of the isolated miR-7704 to the subject.

In some embodiments of the disclosure, the lung injury is an acute lung injury. In another aspect, the lung injury is caused by inflammation. In some embodiments of the disclosure, the lung injury is caused by over expression of a factor of inflammation. Examples of the factor include, but are not limited to, TNF-α, INF-γ, IL-6, IL-1β, and iNOS.

The present disclosure also provides a method for treating and/or ameliorating inflammation in the lung in a subject in need thereof, wherein the method comprises administering an effective amount of isolated microRNA-7704 (miR-7704) or a composition comprising an effective amount of the isolated miR-7704 to the subject.

The present disclosure also provides a method for promoting polarization of macrophages in a subject in need thereof, wherein the method comprises administering an effective amount of isolated microRNA-7704 (miR-7704) or a composition comprising an effective amount of the isolated miR-7704 to the subject.

In some embodiments of the disclosure, the method is for promoting M2 polarization of macrophages.

In some embodiments of the disclosure, the method is for promoting expression of a marker of M2 polarization. Examples of the maker include, but are not limited to, Arg1, Cd206 and IL-10.

In some embodiments of the disclosure, the isolated miR-7704 is obtained from an exosome, an exosome pellet, or physiological solution derived from stem cells.

In some embodiments of the disclosure, the stem cells are mesenchymal stem cells (MSCs). Examples of the stem cells include, but are not limited to umbilical cord mesenchymal stem cells (UMSCs), adipose derived mesenchymal stem cells (ADSCs), or bone marrow mesenchymal stem cells (BMSCs).

In some embodiments of the disclosure, the exosomes are derived from licensed MSCs (LMSCs). In some embodiments of the disclosure, the licensed MSCs are obtained by culturing with INF-α or INF-γ. In another aspect, the exosomes express a maker selected from the group consisting of CD63, CD9 and Alix.

The present disclosure also provides exosomes comprising enriched miR-7704.

In some embodiment, the exosomes are derived from licensed MSCs (LMSCs). In some embodiments of the disclosure, the licensed MSCs are obtained by culturing with INF-α or INF-γ. In another aspect, the exosomes express a maker selected from the group consisting of CD63, CD9 and Alix.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIGS. 1A and 1B show characterization of MSCs and Licensed MSCs. MSCs were treated with TNF-α and IFN-γ for 24 h. FIG. 1A shows representative morphology of MSCs and LMSCs. Left panel scale bar=500 μm; right panel scale bar=100 μm. FIG. 1B shows expression of immuno-suppressive genes (Ido, Pge2, Cxcl10, Cxcl9) were measured by real-time PCR (n=6); results were normalized to the MSCs group. Results are present as the means±SEM. Statistical analyses were performed using Student's two-tailed t-test. (*p<0.05, **p<0.01, *** p<0.001; ns, not significant).

FIGS. 2A to 2C show characterization of exosomes derived from MSCs and Licensed MSCs. FIG. 2A shows transmission electron micrographs of exosomes isolated from MSCs (MSC-exo) and LMSCs (LMSC-exo). Arrow indicates exosomes. Scale bar=200 nm. FIG. 2B shows that Nano particle Tracking Analysis (NTA) revealed the size distribution. FIG. 2C shows that exosome markers CD63, CD9, and Alix were measured by Western blotting analysis. Sup.=Supernatant.

FIGS. 3A to 3B show that RNA sequencing analysis revealed enrichment of miR-7704 in exosomes after licensing. Volcano plot histogram (as shown in FIG. 3A) representative of exosomal miRNA differential expression from MSCs and LMSCs. The abscissa represents fold change (log 2, log 2 FC); ordinates represents significance (−log¹⁰ p value). A log 2 fold change (log 2FC)>1 and a p value<0.05 were used to identify significantly differentially expressed miRNA; red dots represent significantly upregulated miRNA and green dots represent significantly downregulated miRNA. miR-7704 was highlighted in red arrow. FIG. 3B shows that exosomal miRNA expression from MSCs and LMSCs were measured by real-time PCR. Results are present as the means±SEM. Statistical analyses are performed using Student's two-tailed t-test. (*p<0.05, **p<0.01, *** p<0.001; ns, not significant).

FIGS. 4A to 4F show that miR-7704 ameliorated LPS-induced ALI. FIG. 4A shows Schematic showing the experimental design of ALI, control (PBS/PBS), LPS (LPS/PBS), miR-7704 (LPS/miR-7704) and LMSC-exo (LPS/LMSC-exo) administration, and analysis. (Each group N=6). FIG. 4B shows MicroSprayer Aerosolizer. FIG. 4C shows representative H&E staining in different groups. Scale bar=200 μm. FIGS. 4D and 4E show representative IHC (Immunohistochemistry) staining for CD86 (FIG. 4D) and CD206 (FIG. 4E) expression in different groups under 200X (upper lane) and 400X (lower lane) magnification. Brown: positively stained cells. Scale bar=200 μm. FIG. 4F shows inflammatory related gene expression by real-time PCR in different groups. (Each group N=6). Results are present as the means±SEM. Statistical analyses are performed using One-way ANOVA. (*p<0.05, **p<0.01, *** p<0.001; ns, not significant).

FIG. 5A to 5B show that miR-7704 promoted M2 polarization of macrophages. FIG. 5A shows that macrophage surface makers (CD86, CD206) were analyzed by flow cytometry. (Each group n=6). FIG. 5B shows gene expression of M1, M1+miR-7704 and M2. Inflammatory and M1 marker genes (ITNF-α, FN-γ, Il-6); M2 related genes (Arg-1, Cd206, 11-10). (Each group n=6. Results are present as the means±SEM. Statistical analyses are performed using One-way ANOVA. (*p<0.05, **p<0.01, *** p<0.001; ns, not significant).

FIG. 6A shows putative miR-7704 binding sites in 3′UTR of MyD88 mRNA. miR-7704 has been predicted with the binding ability on the MyD88 mRNA.

FIG. 6B shows the results of immunoblotting of cell lysates from macrophages (STAT1, pSTAT1 and MyD88).

### DETAILED DESCRIPTION OF THE INVENTION

Unless defined otherwise, all scientific or technical terms used herein have the same meaning as those understood by persons of ordinary skill in the art to which the present invention belongs. Any method and material similar or equivalent to those described herein can be understood and used by those of ordinary skill in the art to practice the present invention.

The term “a/an” should mean one or more than one of the objects described in the present invention. The term “and/or” means either one or both of the alternatives. The term “a cell” or “the cell” may include a plurality of cells.

The term “and/or” is used to refer to both things or either one of the two mentioned.

The term “isolated” as used herein refers to molecules, cells or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or tissues that are isolated from other cells or tissues and is meant to encompass both cultured and engineered.

As used herein, the terms “microRNA”, “miRNA” and “miR” are used synonymously to refer to an about 18-25 nucleotide (nt) long, non-coding RNAs derived from endogenous genes. “MicroRNA” (miRNA) includes primary miRNA transcripts (pri-miRNA) or other mRNA transcripts that code for mature miRNA (e.g., miRNA processed from introns excised from mRNA transcripts), precursor miRNAs (pre-miRNA), mature single stranded miRNAs, and variants thereof, which may be naturally occurring. The term “miRNA” includes human and miRNA from other mammals. In some instances, the term “miRNA” also includes primary miRNA transcripts and duplex miRNAs. Unless otherwise noted, when used herein, the name of a specific miRNA refers to the mature miRNA of a precursor miRNA. Some single primary miRNA transcripts may contain more than one precursor/mature miRNA. Some mature miRNA may be derived from more than one precursor miRNA.

The term “exosome” refers to cell-derived vesicles having a diameter of between about 20-140 nm, such as between 40 and 120 nm, preferably a diameter of about 50-100 nm, for example, a diameter of about 60 nm, 70 mu, 80 nm, 90 nm, or 100 mm Exosomes may be isolated from any suitable biological sample from a mammal and cultured mammalian cells such as mesenchymal stem cells. As one of skill in the art will appreciate, cultured cell samples will be in the cell-appropriate culture media (using exosome-free serum). Exosomes include specific surface markers not present in other vesicles.

The terms “treatment,” “treating,” and “treat” generally refer to obtaining a desired pharmacological and/or physiological effect. The effect maybe preventive in terms of completely or partially preventing a disease, disorder, or symptom thereof, and may be therapeutic in terms of a partial or complete cure for a disease, disorder, and/or symptoms attributed thereto. “Treatment” used herein covers any treatment of a disease in a mammal, preferably a human, and includes (1) suppressing development of a disease, disorder, or symptom thereof in a subject or (2) relieving or ameliorating the disease, disorder, or symptom thereof in a subject.

The terms “individual,” “subject,” and “patient” herein are used interchangeably and refer to any mammalian subject for whom diagnosis, treatment, or therapy is desired.

The term “effective amount” refers to the amount of exosome, an exosome pellet, or physiological solution that, when administered to a patient or a subject in need for treating a disease or disorder, is sufficient to have a beneficial effect with respect to that disease or disorder. The therapeutically effective amount will vary depending on the conditions of the disease or disorder and its severity. It is not limited to the range stated in the specification. Determining the therapeutically effective amount of given exosome, an exosome pellet, or physiological solution is within the ordinary skill of the art and requires no more than routine experimentation.

As used herein, the term “derived from” shall be taken to indicate that a particular sample or group of samples has originated from the species specified, but has not necessarily been obtained directly from the specified source.

The mesenchymal stem cells according to the disclosure can be obtained from different sources, preferably from umbilical cord, adipose tissue, or bone marrow. According to different sources, the mesenchymal stem cells are umbilical cord mesenchymal stem cells (UMSCs), adipose derived mesenchymal stem cells (ADSCs), and bone marrow mesenchymal stem cells (BMSCs). In some embodiments of this disclosure, MSCs are isolated and isolated from the umbilical cord, and referred to as “umbilical MSC” or “UMSC.” In some embodiments, it is established that the UMSC in this disclosure expresses the same selection of surface markers as the MSC isolated from other bodies, and demonstrates comparable activities.

In another aspect, the present disclosure provides a method for treating and/or ameliorating a lung injury in a subject in need thereof, wherein the method comprises administering isolated microRNA-7704 (miR-7704) or a composition comprising the isolated miR-7704 to the subject. The present disclosure also provides a method for treating and/or ameliorating inflammation in the lung in a subject in need thereof, wherein the method comprises administering an effective amount of isolated microRNA-7704 (miR-7704) or a composition comprising an effective amount of the isolated miR-7704 to the subject.

In some embodiments of the disclosure, miR-7704 has the amino acid sequence of CGGGGUCGGCGGCGACGUG (SEQ ID NO: 1).

In some embodiments of the disclosure, the instant disclosure provides methods and compositions for treatment of acute lung injury, such as but not limited to lung injury resulting from bacterial sepsis, hemorrhagic shock, toxic inhalation, and bleomycin and other drug-induced lung injury. In one embodiment of the pharmaceutical composition, the acute lung injury is an acute respiratory distress syndrome.

In one embodiment of the pharmaceutical composition, the acute lung injury is related to a pulmonary (direct) or an extrapulmonary (indirect) lung injury.

In one embodiment of the pharmaceutical composition, the pulmonary lung injury is selected from the group consisting of inhalation trauma, aspiration trauma, toxic lung edema, lung infection, preferably pneumonia, lung contusion, and embolism.

In one embodiment of the pharmaceutical composition, the extrapulmonary lung damage is associated with a disorder selected from the group consisting of sepsis, polytrauma, shock, burn, acute pancreatitis, drug intoxication, alcohol abuse, chronic lung disease, mass transfusion, disseminated intravascular coagulation, erythema, and autoimmune lung disease.

In some embodiments of the disclosure, the lung injury is caused by inflammation. In some embodiments of the disclosure, the lung injury is caused by over expression of a factor of inflammation. Examples of the factor include, but are not limited to, TNF-α, INF-γ, IL-6, IL-1β, and iNOS.

The present disclosure also provides a method for promoting polarization of macrophages in a subject in need thereof, wherein the method comprises administering an effective amount of an effective amount of isolated microRNA-7704 (miR-7704) or a composition comprising an effective amount of the isolated miR-7704 to the subject.

In some embodiments of the disclosure, the method is for promoting M2 polarization of macrophages.

The term “polarization” is used herein to designate the phenotypic features and the functional features of the macrophages. The phenotype can be defined through the surface markers expressed by the macrophages. The functionality, can be defined for example based on the nature and the quantity of chemokines and/or cytokines expressed, in particular secreted, by the macrophages. Indeed, the macrophages present different phenotypic and functional features depending of their state, either pro-inflammatory M1-type macrophage or anti-inflammatory M2-type macrophage. M2-type macrophages can be characterized by the expression of surface markers such as CD206, CD11b, PD-L1 and CD200R and then secretion of cytokines such as CCL17. In some embodiments of the disclosure, the method is for promoting expression a marker of M2 polarization. Examples of the maker include, but are not limited to, Arg1, Cd206 and IL-10.

In some embodiments of the disclosure, the isolated miR-7704 is obtained from an exosome, an exosome pellet, or physiological solution derived from stem cells.

In some embodiments of the disclosure, the exosomes are derived from licensed MSCs (LMSCs). In some embodiments of the disclosure, the licensed MSCs are obtained by culturing with INF-α or INF-γ. In another aspect, the exosomes express a maker selected from the group consisting of CD63, CD9 and Alix.

A specific process is used to produce the exosome composition of the present disclosure. A desired amount of cells are provided firstly in a method for producing the exo some composition of the present disclosure. In some embodiments of the disclosure, the cells are cultured in a medium for a period of time sufficient to obtain a desired amount of cells. In some embodiments of the disclosure, the cell count is about 1×10⁵ cells/mL to about 1×10⁸ cells/mL; about 2×10⁵ cells/mL to about 8×10⁷ cells/mL; about 4×10⁵ cells/mL to about 6×10⁷ cells/mL; about 6×10⁵ cells/mL to about 4×10⁷ cells/mL; about 8×10⁵ cells/mL to about 2×10⁷ cells/mL; about 1×10⁶ cells/mL to about 1×10⁷ cells/mL; about 2×10⁶ cells/mL to about 8×10⁶ cells/mL; or about 4×10⁶ cells/mL to about 6×10⁶ cells/mL. The manners for culture depends on the cells. In some embodiments of the disclosure, the medium is a conditioned medium for obtaining the desired amount of cells with specific properties. For example, a conditioned medium is provided for maintaining cells at an undifferentiated stage or a differentiated stage.

The medium containing the desired amount of cells is subjected to a pre-clearance procedure for removing dead cells and/or cell debris. In one embodiment of the disclosure, the medium is centrifuged to remove dead cells. In some embodiments of the disclosure, dead cells can be removed at about 300 g, about 350 g, or about 400 g for about 5 minutes, about 6 minutes, about 7 minutes, about 8 minutes, about 9 minutes, about 10 minutes, about 11 minutes, about 12 minutes, about 13 minutes, about 14 minutes, or about 15 minutes. In one embodiment of the disclosure, the medium is centrifuged to remove cell debris. In some embodiments of the disclosure, cell debris can be removed at about 1500 g, about 1600 g, about 1700 g, about 1700 g, about 1800 g, about 1900 g, about 2000 g, about 2100 g, about 2200 g, about 2300 g, about 2400 g, or about 2500 g, for about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

The resulting supernatant after removing the dead cells and/or cell debris is then filtrated to remove cell apoptotic bodies and microvesicles. In one embodiment of the disclosure, the resulting supernatant is filtrated by passing through about 0.15 μm, about 0.16 μm, about 0.18 μm, about 0.20 μm, about 0.22 μm, about 0.24 μm, about 0.26 μm, or about 0.28 μm filter.

A type of membrane filtration in which forces (such as pressure or concentration gradients) lead to a separation through a semipermeable membrane. Ultrafiltration membranes are typically characterized by the molecular weight cut off of the membrane. Suspended solids and solutes of higher molecular weight are retained in the retentate, while water and lower molecular weight solutes pass through the membrane in the permeate. Different types of modules can be used for ultrafiltration processes. Examples of such modules are tubular elements that use polymeric membranes cast on the inside of plastic or paper tubes; hollow fiber designs that contain multiple hollow fibers; spiral wound modules in which flat membrane sheets are separated by a thin meshed spacer material that is rolled around a central perforated tube and fitted into a tubular steel pressure vessel casing; and plate and frame assemblies that use a membrane placed on a flat plate separated by a mesh like material through which the filtrate passes.

The resulting supernatant after ultrafiltration is then subjected to exosome precipitation for enrichment. In some embodiments of the disclosure, the resulting supernatant after ultrafiltration is precipitation with polymer-based precipitation. The resulting supernatant comprises a population of enriched exosomes in a concentration greater than about 1×10⁹ exosomes/mL.

In some embodiments of the disclosure, the exosomes are pelleted by centrifugation, e.g. at 10,000×g for 10 min at 4° C. to obtain the exosome pellet. In some embodiments of the disclosure, the exosome pellet is solubilized in a suitable saccharide solution, such as a trehalose solution, that is effective to reduce aggregation of the exosomes.

The pharmaceutical compositions disclosed herein can be administered by one of many routes, depending on the embodiment. For example, exosome administration may be by local or systemic administration. Local administration, may in some embodiments be achieved by direct administration to a tissue (e.g., direct injection, such as intramyocardial injection). Local administration may also be achieved by, for example, lavage of a particular tissue (e.g., intra-intestinal or peritoneal lavage). In several embodiments, systemic administration is used and may be achieved by, for example, intravenous and/or intra-arterial delivery. In certain embodiments, intracoronary delivery is used. In several embodiments, the exosomes are specifically targeted to the damaged or diseased tissues. In some such embodiments, the exosomes are modified (e.g., genetically or otherwise) to direct them to a specific target site.

The administration of the pharmaceutical composition may be performed more than one time; preferably, the pharmaceutical composition are administrated to the subject twice in a time interval. The interval between the administrations may be 1, 2, 3, 4, 5, 6, 7, 8, 9, 10, 11, 12, 13, or 14 days, preferably, 7 days. In some embodiments of the disclosure, the exosome, exosome pellet, or physiological solution is administered every other days.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### Examples

Human MSCs Culture and Characterization

Human mesenchymal stromal cells (hMSCs) are derived from human adipose purchased from Steminent Biotherapeutic Inc. (Taipei, Taiwan). hMSCs were then cultured in Iscove's Modified Dulbecco's modified medium (IMDM; Sigma-Aldrich, St. Louis, MO, USA) supplemented with 10% fetal bovine serum (ES Cell-Qualified FBS; Thermo Fisher Scientific, Waltham, MA, USA) and 1% penicillin-streptomycin-glutamine (PSG; Thermo Fisher Scientific) and FGF2 10 ng/ml (Sigma, SI-F0291). RAW264.7 were culture in low-glucose Dulbecco's modified Eagle's medium (LG-DMEM; Sigma-Aldrich, St. Louis, MO, USA) supplemented with 10% fetal bovine serum (FBS; Thermo Fisher Scientific, Waltham, MA, USA) and 1% penicillin-streptomycin-glutamine (PSG; Thermo Fisher Scientific).

Preparation of Licensed MSCs (LMSCs)

hMSCs were maintained in medium in an appropriate cell density (80% confluency) and incubated with serum-free medium in the presence of TNF-α (20 ng/ml, Sigma Aldrich, SRP3177) and IFN-γ (20 ng/ml, Sigma Aldrich, SRP3058) for 24 hr at 37° C., 5% CO2.

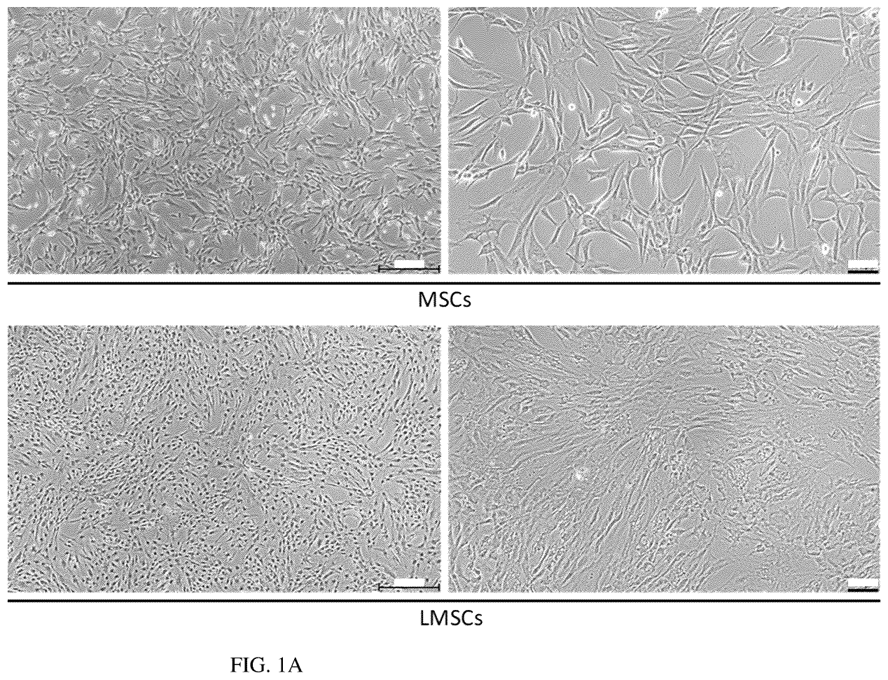

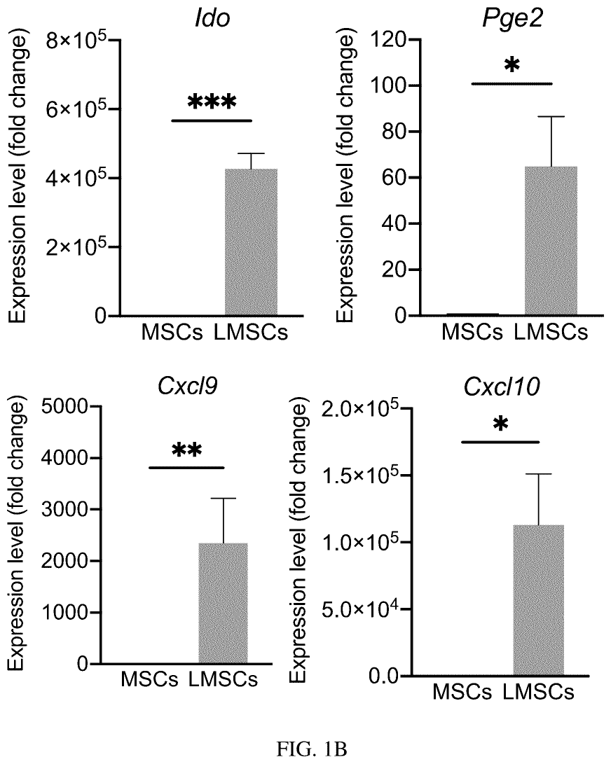

The morphology of MSCs and LMSCs is shown in FIG. 1A. The results of expression of immune-suppressive genes are shown in FIG. 1B. After licensing, the MSCs express a large amount of immune-suppressive genes.

Exosome Isolation and Characterization

MSCs were cultured until optimal confluency with or without TNF-α and IFN-γ. The culture condition was changed with IMDM supplemented with 10% exosome-depleted FBS and 1% PSG. The conditioned medium (CM) was than collected after 24 h of incubation. The cell debris was removed using ultracentrifugation 3000 g, 30 min, 4° C. following by filtering 0.22-μm PVDF filters. Isolation of exosome from MSCs and LMSCs was performed by kit-based method or ultracentrifugation. For kit-based method: MSC-CM or LMSC-CM was concentrated by a 3 kDa Vivaspin concentrator (GE Healthcare, Chicago, IL, USA), and then add Exoprep (Hansa BioMed, Tallinn, Estonia) for exosome isolation following by the manufacture's protocols. For ultracentrifugation, the CM was centrifugated for 100,000 g, 70 min, 4° C., and the exosome pellet was re-suspend with PBS or Trizol for RNA isolation. The exosomes were also identified using western blotting, Nano Particle Tracking Analysis (NanoSight, NTA 3.1 Build 3.1.45) and transmission electronic microscopy (TEM) (HITACHI HT7700, Tokyo, Japan) was performed.

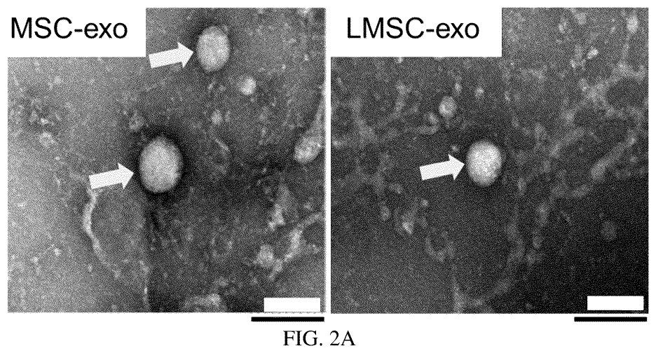

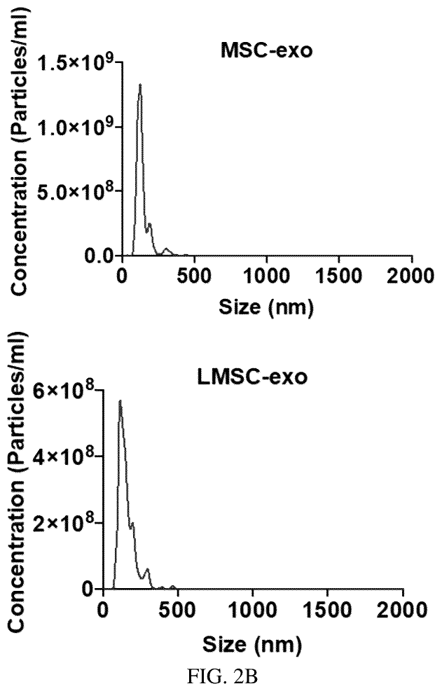

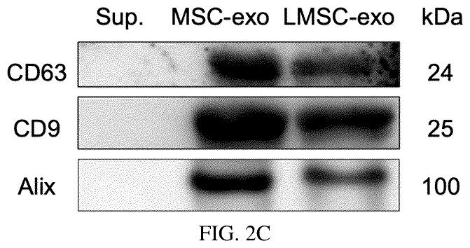

The results are shown in FIGS. 2A to 2C. The electron microscopic view (FIG. 2A) of exosomes shows that the structure is as a round shape, and the size is less than 200 nm. The particle size is less than 200 nm (FIG. 2B). Furthermore, the exosomes exhibit specific markers of, CD63, CD9, and Alix.

Micro-RNA Sequencing and Quantitative Real-Time PCR

Micro-RNA from exosome were isolated by Trizol, according to the manufacturer instruction. RNA samples with an OD260/OD230 ratios around 1.8, OD260/OD280 ratios around 2.0, and RNA integrity≥eight were subjected to small RNA sequencing on an Illumina HiSeq 2500. Transcript abundance of miRNAs between each sample was normalized with transcripts per million (TPM). We conducted differential expression (DE) analysis with limma package in R 3.6.3 (RStudio Team (2020)). RStudio: Integrated Development for R. RStudio, Inc., Boston, MA URL http://www.rstudio.com/) to identify significantly DE miRNAs according to the thresholds of the absolute value of log 2 fold-change (log 2 FC)>1 and a P value<0.05.

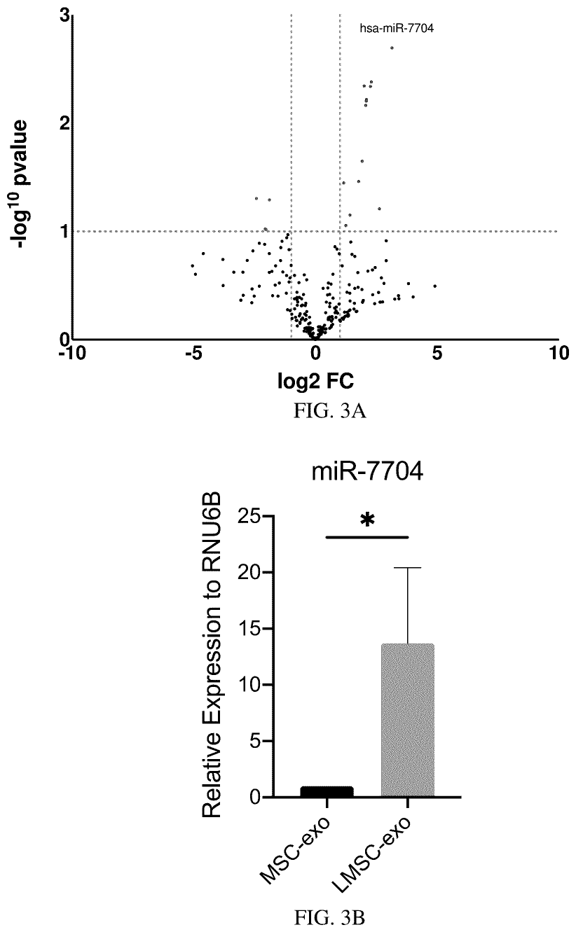

The results are shown in FIGS. 3A to 3C. miR-7704 greatly express in the exosomes of LMSCs.

LPS-Induced Acute Lung Injury Mouse Model

Eight-to-ten-week male C57BL/6JNarl mice. After two weeks of acclimatization, the mice were randomly divided into four groups (n=6), control (PBS/PSB), LPS (LPS/PBS), mir-7704 (LPS/miR-7704), exosome from Licensed MSCs (LPS/exosome). The mice were subjected to 20 mg/kg of Lipopolysaccharide (LPS from Escherichia coli O111:B4, Sigma L2630) via intratracheally administration to generate ALI model. 10 h after LPS injury, 200 nM of miR-7704 or 50 ug of LMSC-exo was also administrated through intratracheally injection. All groups was slowly injected into the lungs using a MicroSprayer Aerosolizer (Shanghai Yuyan Instruments Co., Ltd, China, YAN30012). 24 h after LPS injury, mice were euthanized by over-dosed isoflurane inhalation. The lung tissue were taken for analysis.

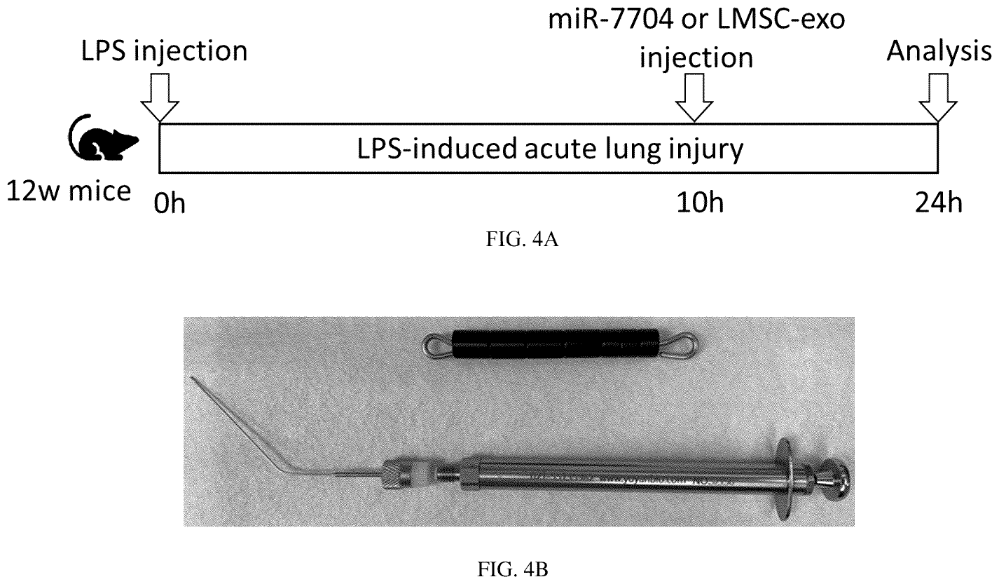

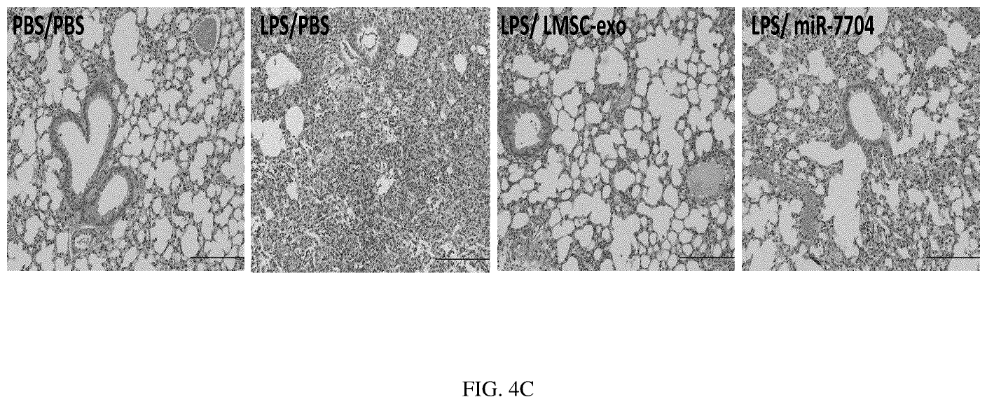

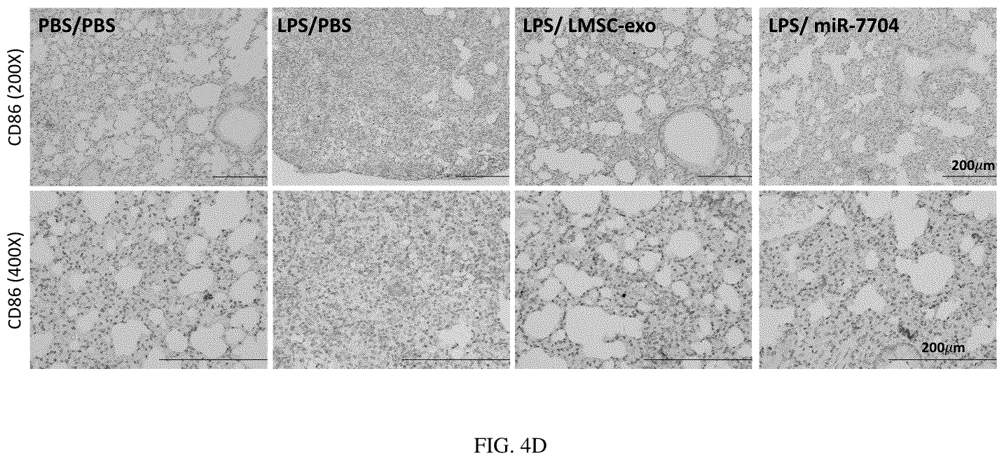

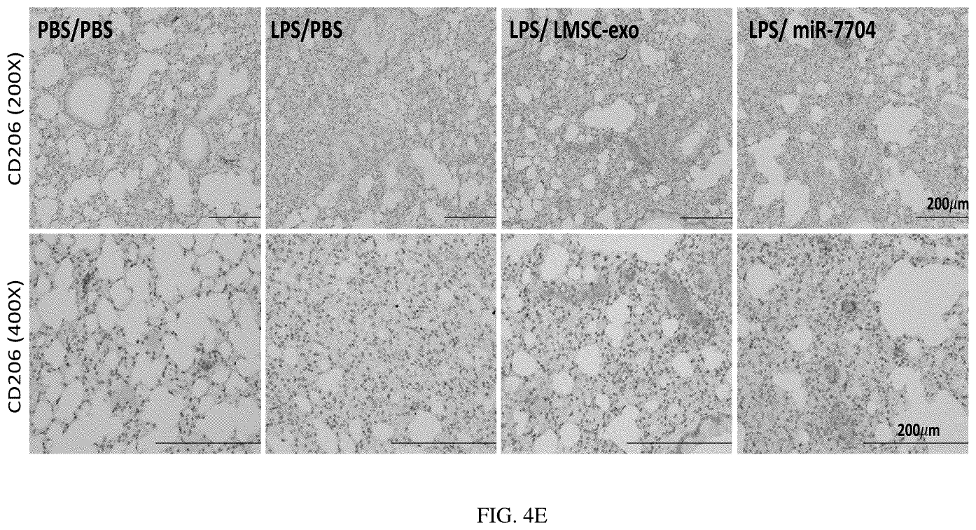

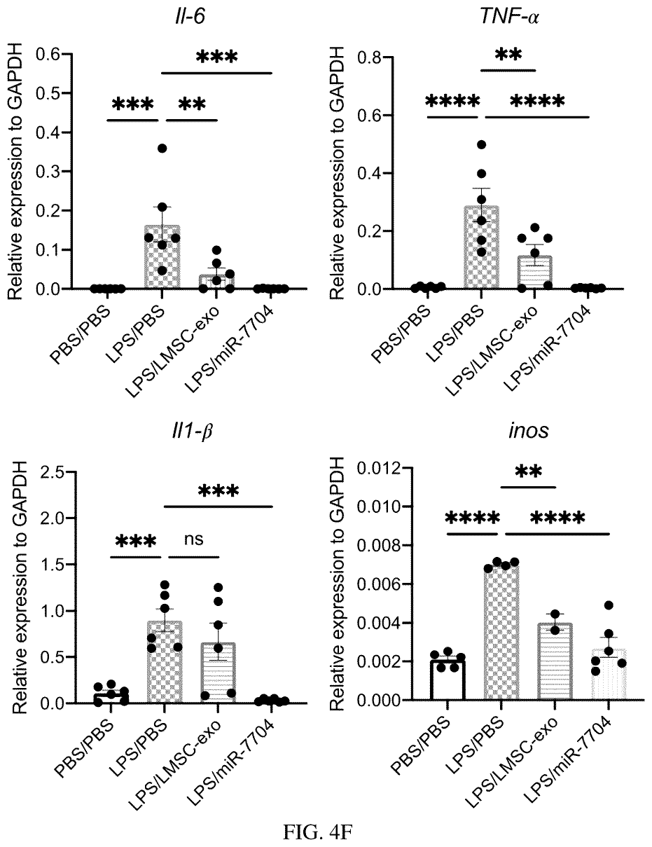

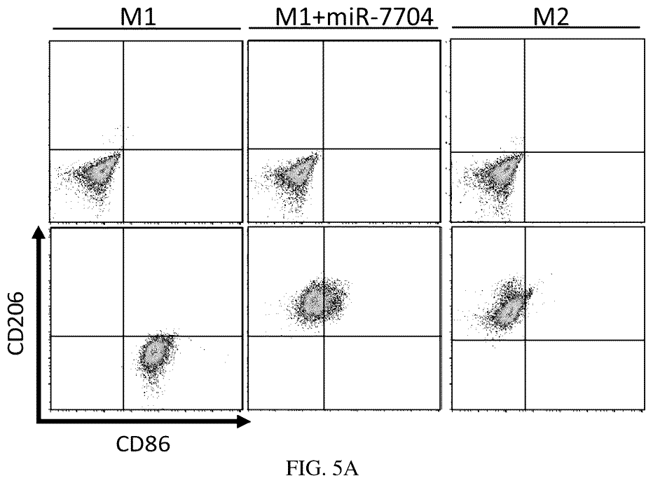

The results are shown in FIGS. 4A to 4F. The results of immunohistology staining show increased CD86 positive cells and decreased CD206 positive cells in lung tissue with ALI (LPS/PBS) compared to normal lung tissue (PBS/PBS), indicating M1 macrophages are dominant in an injured lung tissue. Furthermore, both miR-7704 (LPS/miR-7704) and LMSC-exo (LPS/exosome) decreased CD86 positive cells and increased CD206 positive cells in lung tissue with ALI supporting that M2 macrophages are dominant after miR-7704 or LMSC-exo treatment.

Micro-RNA Transfection in Cells

Mouse BMDMs were seeded into 6 cm dish at a density of 1×10⁶ cell/well, then the transfection of miRNA was conducted with a miR-7704 mimic (100 nM) (Biotools, TW) using TOOLSmartFect Transfection Reagent (Biotools, TN-S01,TW) following by the manufacturer's protocol. In brief, miR-7704 mimic and the transfection reagent are mixed with basal LG-DMEM medium (Thermo Fisher Scientific, 11875093) without FBS as described previously, and the transfection complex are formed after 30 min of incubation in RT. The miRNA complex are transfected into cells for 24 hr and the transfection efficiency and efficacy are conducted by quantitative real-time PCR, flow cytometry, ELISA and Western blotting.

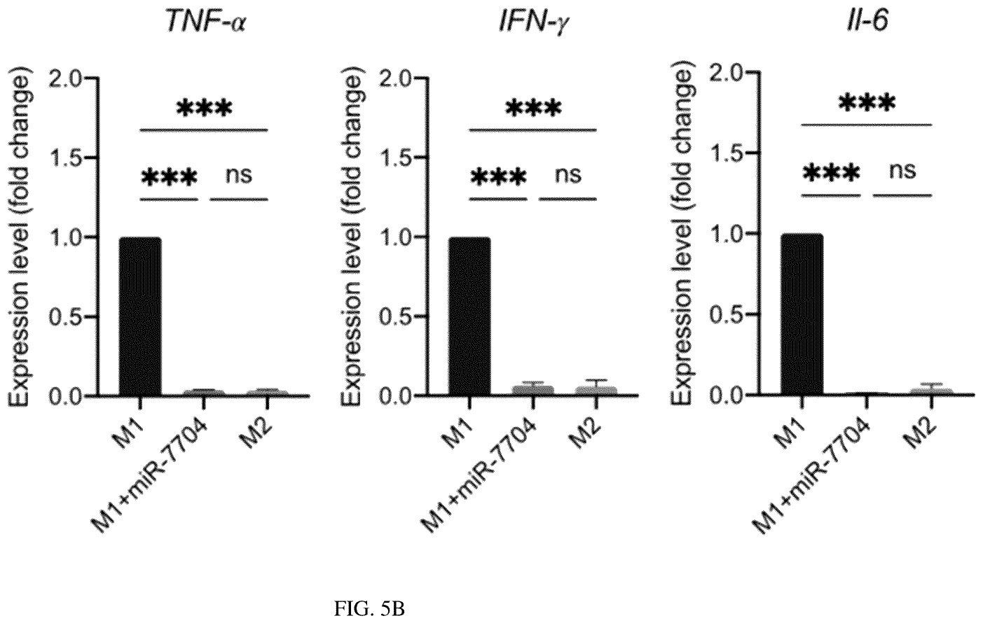

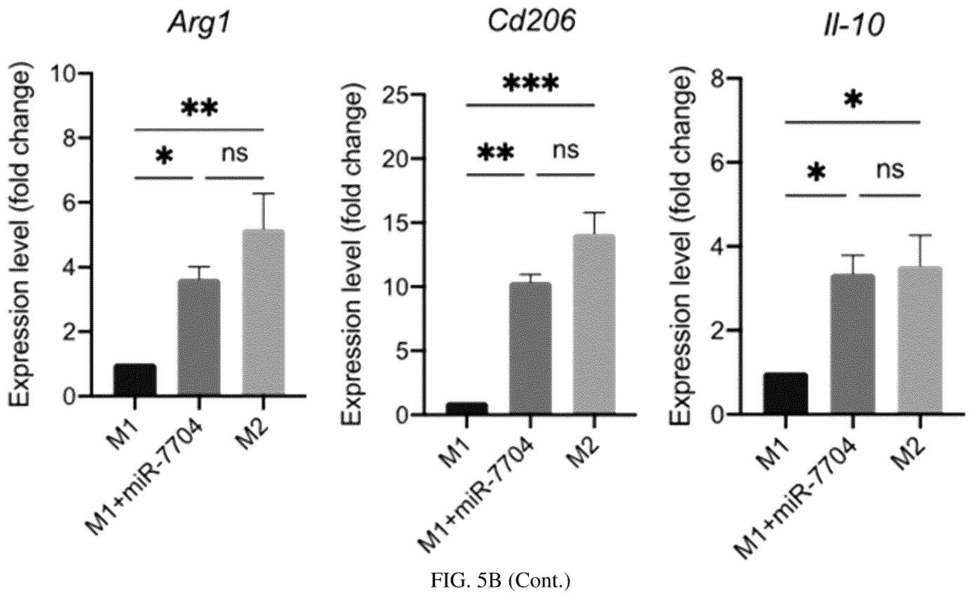

The results are shown in FIGS. 5A and 5B. MiR-7704 promotes M2 polarization in macrophages. These findings suggest that miR-7704 plays a key role in promoting M2 polarization in macrophages.

MiR-7704 Promotes M2 Polarization Through MyD88/STAT1 Inhibition

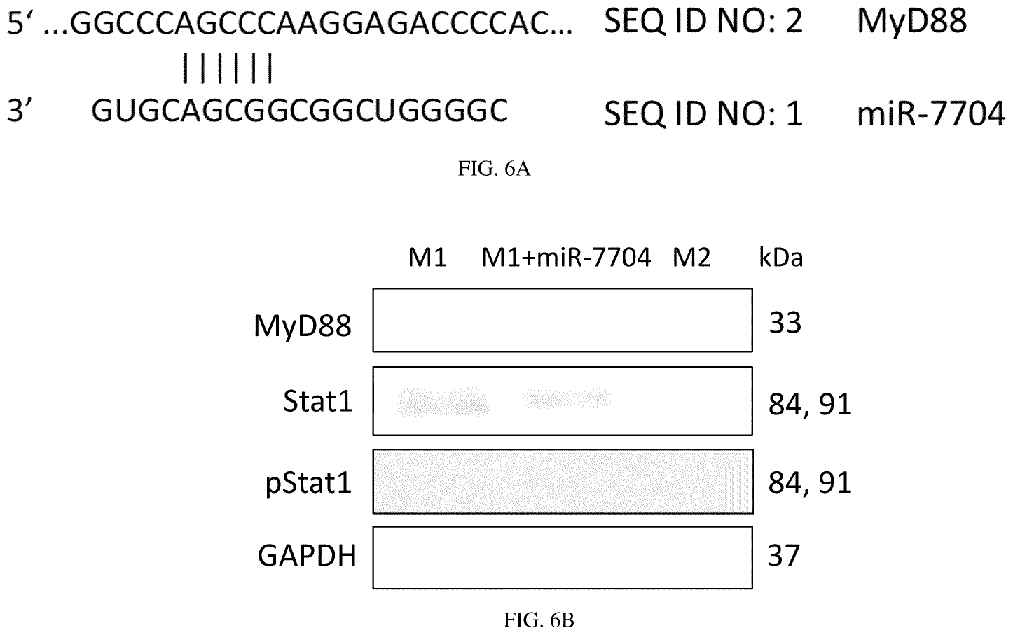

The putative miR-7704 binding sites in 3′UTR of MyD88 mRNA is predicted and shown in FIG. 6A. The effect of MiR-7704 on M2 polarization was also assayed by immunoblotting. The results are shown in FIG. 6B. MyD88 expression was inhibited in miR-7704 transfected M1 macrophages STAT1 was identified with significantly downregulated and inflammatory pathways in miR-7704 transfected macrophages. In addition, both STAT1 and phosphorylated STAT1 were significantly abolished in M1 macrophages after miR-7704 transfection miR-7704 affects phenotypic alternation of macrophages towards to M2 polarization through inhibiting MyD88/STAT1. The results support the notion that miR-7704 affects the phenotypic alternation of macrophages toward M2 polarization by inhibiting MyD88/STAT1.

While the present disclosure has been described in conjunction with the specific embodiments set forth, many alternatives thereto and modifications and variations thereof will be apparent to those of ordinary skill in the art. All such alternatives, modifications and variations are considered to fall within the scope of the present disclosure.

What is claimed is:

1. A method for treating and/or ameliorating a lung injury or inflammation in a subject in need thereof, wherein the method comprises administering an effective amount of isolated microRNA-7704 (miR-7704) or a composition comprising an effective amount of the isolated miR-7704 to the subject.

2. The method of claim 1, wherein the lung injury is an acute lung injury.

3. The method of claim 1, wherein the lung injury is caused by inflammation.

4. The method of claim 1, wherein the lung injury is caused by over expression of a factor selected from the group consisting of TNF-α, INF-γ, IL-6, IL-1β, and iNOS.

5. The method of claim 1, wherein the method is for treating and/or ameliorating a lung injury or inflammation through promotion of polarization of macrophages.

6. The method of claim 5, wherein the method is for promoting M2 polarization of macrophages.

7. The method of claim 5, wherein the method is for promoting expression a marker selected from the group consisting of Arg1, Cd206 and IL-10.

8. The method of claim 1, wherein the isolated miR-7704 is obtained from an exosome, an exosome pellet, or physiological solution derived from stem cells.

9. The method of claim 8, wherein the stem cells are mesenchymal stem cells (MSCs).

10. The method of claim 8, wherein the stem cells are umbilical cord mesenchymal stem cells (UMSCs), adipose derived mesenchymal stem cells (ADSCs), or bone marrow mesenchymal stem cells (BMSCs).

11. The method of claim 8, wherein the exosomes are derived from licensed MSCs (LMSCs).

12. The method of claim 11, wherein the licensed MSCs are obtained by culturing with INF-α or INF-γ.

13. The method of claim 8, wherein the exosomes express a maker selected from the group consisting of CD63, CD9 and Alix.

14. An exosome comprising enriched miR-7704.

15. The exosome of claim 14, which is derived from stem cells.

16. The exosome of claim 15, wherein the stem cells are umbilical cord mesenchymal stem cells (UMSCs), adipose derived mesenchymal stem cells (ADSCs), or bone marrow mesenchymal stem cells (BMSCs).

17. The exosome of claim 14, wherein the exosomes are derived from licensed MSCs (LMSCs).

18. The exosome of claim 14, wherein the licensed MSCs are obtained by culturing with INF-α or INF-γ.

19. The exosome of claim 14, wherein the exosomes express a maker selected from the group consisting of CD63, CD9 and Alix.
